(19) United States
(12) Patent Application Publication
(10) Pub. No.: US 2013/0158632 A1
(43) Pub. Date: Jun. 20, 2013

(54) METHODS OF MANUFACTURING TARGET FOR GENERATING CHARGED PARTICLES, TARGET STRUCTURES, AND TREATMENT APPARATUSES INCLUDING THE TARGET STRUCTURES

(71) Applicant: Electronics and Telecommunications Research, Daejeon (KR)

(72) Inventors: Hyung Ju PARK, Suwon (KR); Hyeon-Bong PYO, Daejeon (KR); Seong-Mok CHO, Daejeon (KR); Moon Youn JUNG, Daejeon (KR); Seunghwan KIM, Daejeon (KR)

(73) Assignee: ELECTRONICS AND TELECOMMUNICATIONS RESEARCH INSTITUTE, Daejeon (KR)

(21) Appl. No.: 13/687,177

(22) Filed: Nov. 28, 2012

(30) Foreign Application Priority Data
Dec. 19, 2011 (KR) 10-2011-0137392

(51) Int. Cl. A61N 5/02

(52) U.S. Cl. CPC A61N 5/022
USPC 607/89; 205/122; 427/261

(57) ABSTRACT

Provided are a method of manufacturing a target for generating charged particles, a target structure, and a treatment apparatus including the target structure. The method includes forming a source layer on a substrate, forming balls on the source layer, forming a metal thin layer on the source layer exposed between the balls, and removing the balls to form holes in the metal thin layer so as to expose the source layer.

### CROSS-REFERENCE TO RELATED APPLICATIONS

This U.S. non-provisional patent application claims priority under 35 U.S.C. §119 of Korean Patent Application No. 10-2011-0137392, filed on Dec. 19, 2011, the entire contents of which are hereby incorporated by reference.

### BACKGROUND OF THE INVENTION

The present invention disclosed herein relates to a target and a method of manufacturing the target, and more particularly, to a method of manufacturing a target for generating charged particles, a target structure, and a treatment apparatus including the target structure.

Unlike X-ray or gamma ray treatment methods, treatment methods using charged particles accurately remove tumor cells with minimizing damage to normal tissues, and thus are regarded with much interest as patient-friendly treatment methods. However, recent treatment apparatuses using charged particles require not only a large-sized device for generating charged particles, but also high installation and maintenance costs. To address these limitations, charged particle generating methods using a high power pulse laser are suggested, thereby significantly decreasing the size and cost of treatment apparatuses.

Competitive laser ion accelerators are required to generate charged particles of high energy so as to treat a tumor located deeply inside the body However, most of recent methods just increase the intensity of a laser source in order to generate charged particles of high energy.

### SUMMARY OF THE INVENTION

The present invention provides a method of manufacturing a target capable of generating high energy charged particles, a target structure, and a treatment apparatus including the target structure.

The present invention also provides a method of manufacturing a target for generating charged particles, a target structure, and a treatment apparatus including the target structure, which improves or maximizes productivity.

Embodiments of the inventive concept provide methods of manufacturing a target of a treatment apparatus, including: forming a source layer on a substrate; forming balls on the source layer; forming a metal thin layer on the source layer exposed between the balls; and removing the balls to form holes in the metal thin layer so as to expose the source layer.

In some embodiments, the forming of the balls may include a colloid lithograph process.

In other embodiments, the balls may include at least one of a block copolymer and an amphiphilic surfactant.

In still other embodiments, the balls may be removed by a volatile solvent such as alcohol.

In even other embodiments, the source layer may include at least one of silicon, a dielectric, and a carbon thin layer.

In yet other embodiments, the dielectric may include a metal oxide layer, a silicon oxide layer, or a silicon nitride layer.

In further embodiments, the forming of the metal thin layer may include a sputtering process or an electroplating process.

In still further embodiments, the metal thin layer may include at least one of gold, silver, and copper.

In other embodiments of the inventive concept, targets include: a substrate; a source layer disposed on the substrate; and a metal thin layer including holes partially exposing the source layer.

In some embodiments, the metal thin layer may include a precious metal such as gold, silver, or copper.

In other embodiments, the holes of the metal thin layer may have a size ranging from about 1 nm to about 1 μm.

In still other embodiments, the substrate may include a window that exposes the source layer or the metal thin layer to a lower side thereof.

In even other embodiments, the window may be provided in plurality to be arrayed in a matrix or in concentric circles within the substrate.

In yet other embodiments, the source layer may include at least one of silicon, a dielectric, and a carbon thin layer.

In further embodiments, the dielectric may include at least one of a metal oxide layer, a silicon oxide layer, and a silicon nitride layer.

In still further embodiments, the carbon thin layer may include at least one of graphene, graphite, fullerene, and a carbon nanotube.

In still other embodiments of the inventive concept, treatment apparatuses include: a light source providing a laser beam; a guiding structure disposed at an incidence side of the laser beam provided by the light source; and a target including a substrate fixed to the guiding structure, a source layer disposed on the substrate, and a metal thin layer including holes partially exposing the source layer, wherein the metal thin layer accelerates, through surface plasmon resonance, charged particles generated in the source layer by the laser beam passing through the holes.

In some embodiments, the light source may include a microwave laser.

In other embodiments, the metal thin layer may include a precious metal such as gold, silver, or copper.

### BRIEF DESCRIPTION OF THE DRAWINGS

The accompanying drawings are included to provide a further understanding of the present invention, and are incorporated in and constitute a part of this specification. The drawings illustrate exemplary embodiments of the inventive concept and, together with the description, serve to explain principles of the present invention. In the drawings:

FIG. 1 is a cross-sectional view illustrating a tumor treatment apparatus using a target for generating charged particles according to an embodiment of the inventive concept;

FIG. 2 is a plan view illustrating the target of FIG. 1;

FIGS. 3A and 3B are schematic views illustrating a tumor treatment process using the charged particles of FIG. 1;

FIGS. 4 to 8 are cross-sectional views illustrating a method of manufacturing a target according to another embodiment of the inventive concept; and

FIGS. 9 to 11 are cross-sectional views illustrating a method of manufacturing a target according to another embodiment of the inventive concept.

### DETAILED DESCRIPTION OF PREFERRED EMBODIMENTS

Embodiments of the inventive concept will be described below in detail with reference to the accompanying drawings. The present invention may, however, be embodied in different forms and should not be construed as limited to the embodiments set forth herein. Rather, these embodiments are provided so that this disclosure will be thorough and complete, and will fully convey the scope of the present invention to those skilled in the art. Like reference numerals refer to like elements throughout.

In the following description, the technical terms are used only for explaining specific exemplary embodiments while not limiting the present invention. The terms of a singular form may include plural forms unless referred to the contrary. The meaning of ‘comprises’ and/or ‘comprising’ specifies a property, a region, a fixed number, a step, a process, an element and/or a component but does not exclude other properties, regions, fixed numbers, steps, processes, elements and/or components. Since exemplary embodiments are provided below, the order of the reference numerals given in the description is not limited thereto. In the specification, it will be understood that when a layer (or film) is referred to as being ‘on’ another layer or substrate, it can be directly on the other layer or substrate, or intervening layers may also be present.

Additionally, the embodiments in the detailed description may be described with cross-sectional views and/or plan views as ideal exemplary views of the present invention. In the figures, the dimensions of layers and regions are exaggerated for clarity of illustration. Accordingly, shapes of the exemplary views may be modified according to manufacturing techniques and/or allowable tolerances. Therefore, the embodiments of the inventive concept are not limited to the specific shape illustrated in the exemplary views, but may include other shapes that may be created according to manufacturing processes. For example, an etched region illustrated as a rectangle may have rounded or curved features. Thus, areas exemplified in the drawings have general properties, and are used to illustrate a specific shape of a device region. Accordingly, this should not be construed as limited to the scope of the present invention.

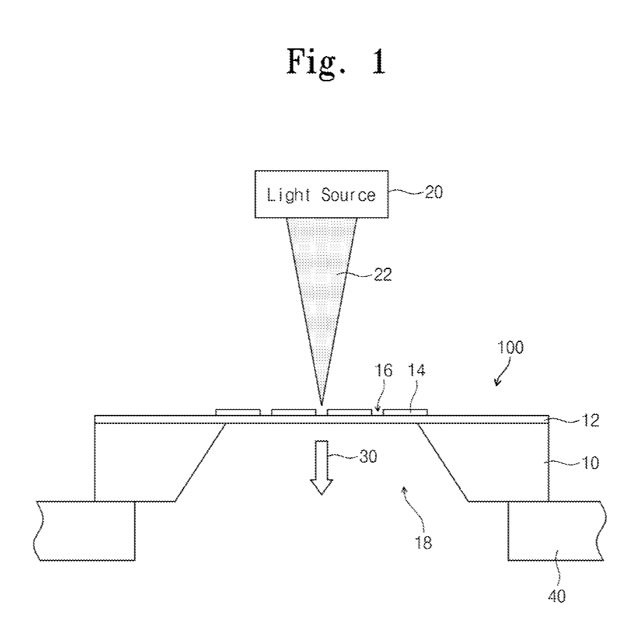

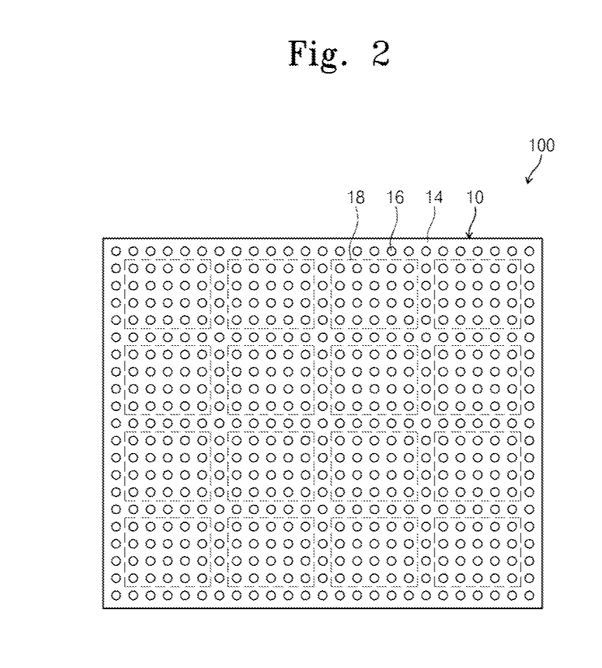

FIG. 1 is a cross-sectional view illustrating a tumor treatment apparatus using a target for generating charged particles according to an embodiment of the inventive concept. FIG. 2 is a plan view illustrating the target of FIG. 1.

Referring to FIGS. 1 and 2, a treatment apparatus according to the current embodiment may include a target 100 that uses a laser beam 22 to generate charged particles 30 of high energy. The target 100 may include: a source layer 12 disposed on a substrate 10; and a metal thin layer 14 including nanoholes 16 to partially expose the source layer 12. The laser beam 22 has high energy by plasmon resonance through the nanoholes 16. The source layer 12 may emit charged particles by using the laser beam 22. A light source 20 may include a microwave laser for generating the laser beam 22.

Thus, the treatment apparatus generates the charged particles 30 of high energy proportional to an intensity of the laser beam 22 incident to the source layer 12.

The substrate 10 may be fixed by a guard ring structure 40. The guard ring structure 40 may include a holder for fixing the substrate 10. The substrate 10 may include windows 18 to expose the source layer 12 to the lower side thereof. The source layer 12 may include silicon, a dielectric thin layer, or a carbon thin layer to generate the charged particles 30. The dielectric thin layer may include a metal oxide layer, a silicon oxide layer, a metal nitride layer, or a silicon nitride layer. The carbon thin layer may include graphene, graphite, fullerene, or carbon nanotubes. The thickness of the source layer 12 may be varied according to the characteristics of an incident laser beam.

The source layer 12 may include a grid in the windows 18 to expose the metal thin layer 14 to the lower side thereof. The grid may include a metal or silicon material. The grid may include a mesh having holes to expose the metal thin layer 14 to the lower side thereof. The grid may be used for a transmission electron microscope (TEM). The substrate 10 may include a silicon wafer or silicon bar including the windows 18. The windows 18 may be arrayed in a matrix within the silicon bar. Although not shown, the silicon wafer may be a disk including the windows 18 arrayed in concentric circles.

The metal thin layer 14 may include a precious metal such as gold (Au), silver (Ag), or copper (Cu). The metal thin layer 14 may have a thickness ranging from about 1 nm to about 1 μm. The nanoholes 16 of the metal thin layer 14 may have a size ranging from about 1 nm to about 800 nm. The nanoholes 16 may be spaced apart from one another by a distance ranging from about 10 nm to about 1 μm above the substrate 10. The nanoholes 16 may be arrayed regularly or randomly.

As described above, the laser beam 22 may be accelerated to high energy within the nanoholes 16 of the metal thin layer 14. Surface plasmon may be defined as a near field formed on a surface of the metal thin layer 14 by the laser beam 22. The near field is formed by an interaction between a high density charge distribution of the surface of the metal thin layer 14 and photons that exist along the interface between the metal thin layer 14 and the source layer 12 as a dielectric. The laser beam 22 accelerated within the nanoholes 16 may generate the charged particles 30 in the source layer 12. The charged particles 30 may have energy proportional to output power of the laser beam 22. Thus, the target 100 emits the charged particles 30 of high energy by the laser beam 22.

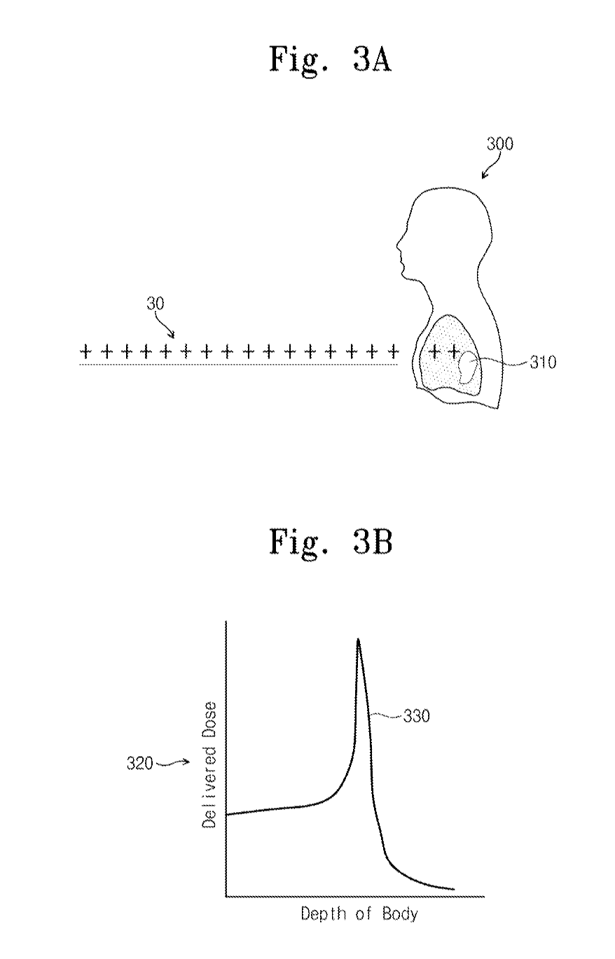

FIG. 3 is a schematic view illustrating a tumor treatment process using the charged particles of FIG. 1.

Referring to FIGS. 1 and 3, the charged particles 30 from the target 100 may travel in the same direction as that of the laser beam 22. The charged particles 30 may be collected in a tumor 310 of a human body 300. The charged particles 30 may be collected to a density corresponding to a Bragg peak value 330 of a graph 320. The charged particles 30 may be collected to a high density in the tumor 310. The charged particles 30 may pass through organs of the human body 300 in proportion to the energy of the laser beam 22. A horizontal axis of the graph 320 denotes depth of the human body 300, and a vertical axis thereof denotes delivered dose of the charged particles 30. Although not shown, the tumor 310 may be detected by X-ray imaging, magnetic resonance imaging (MRI), computer tomography (CT), positron emission tomography (PET), or a detecting device such as an ultrasonic wave device. The growth of tumor cells may be stopped by the charged particles 30, or the tumor cells may be died thereby.

Thus, commercial viability of the treatment apparatus can be improved or maximized.

A method of manufacturing the target 100 will now be described according to other embodiments of the inventive concept.

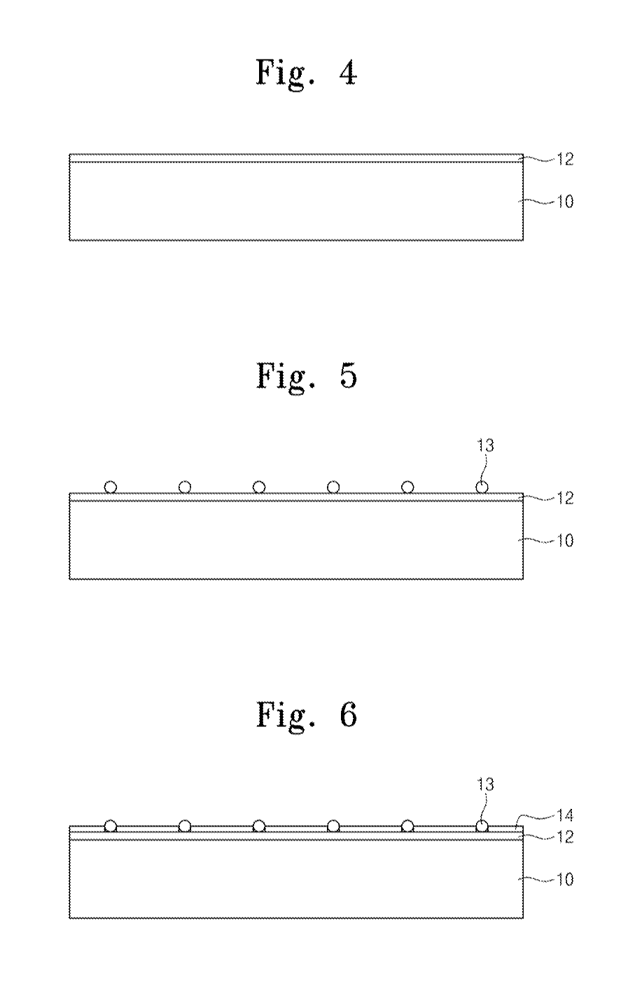

FIGS. 4 to 8 are cross-sectional views illustrating a method of manufacturing a target according to an embodiment of the inventive concept.

Referring to FIG. 4, the source layer 12 is formed on the substrate 10. The substrate 10 may include a bulk type silicon wafer or silicon bar. The source layer 12 may include a dielectric thin layer such as a silicon oxide layer or a silicon nitride layer. The source layer 12 may include a grid (not shown) formed by patterning the dielectric thin layer.

Referring to FIG. 5, sacrificial balls 13 are formed on the source layer 12. The sacrificial balls 13 may be formed through a colloid lithography process. The colloid lithography process is a self-assembly process in which nano structures are spontaneously formed by the covalent bonds between atoms or the attractive forces between molecules. The sacrificial balls 13 may include amphiphilic molecules such as a block copolymer or an amphiphilic surfactant. That is, the sacrificial balls 13 which are nano scale structures may be formed through self assembling of the colloid lithography process.

Referring to FIG. 6, the metal thin layer 14 is formed on the source layer 12 exposed between the sacrificial balls 13. The metal thin layer 14 may include a precious metal such as gold (Au), silver (Ag), or copper (Cu) through a sputtering or electroplating process.

Referring to FIG. 7, the sacrificial balls 13 are removed to form the nanoholes 16 on the metal thin layer 14. The sacrificial balls 13 may be removed by an alcohol or volatile solution. The nanoholes 16 may be formed by performing a cleaning or etching process on the sacrificial balls 13. The nanoholes 16 may be formed through one of a colloid lithography process, a cleaning process, and an etching process, which are more economical than an electron-beam lithography process or a photolithography process.

Accordingly, the method according to the current embodiment can improve or maximize target productivity.

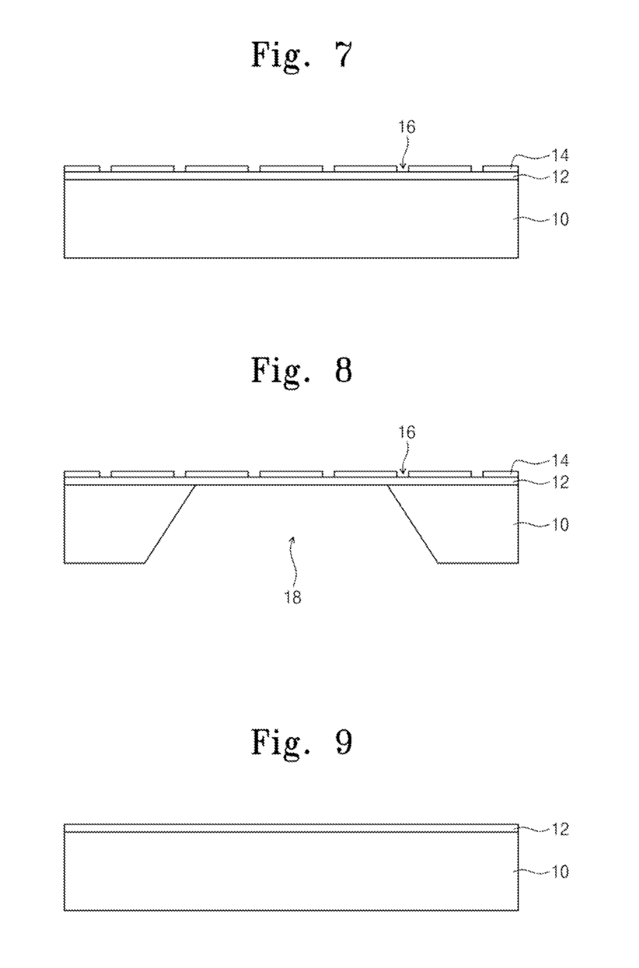

Referring to FIG. 8, a portion of the substrate 10 disposed under the metal thin layer 14 is removed to form the window 18 that partially exposes the source layer 12. The window 18 may be formed through an electron-beam lithography process, an imprint lithography process, a photolithography process, or an etching process. The imprint lithography process or the photolithography process forms an imprint resist pattern (not shown) or a photoresist pattern (not shown), which selectively exposes the bottom surface of the substrate 10 to correspond to the window 18. The etching process selectively removes a portion the substrate 10 exposed through a photoresist pattern. The electron-beam lithography process selectively removes a portion of the substrate 10 remaining the bottom surface of the source layer 12 while the window 18 is formed.

As a result, in the method according to the current embodiment, the nanoholes 16 of the metal thin layer 14 can be formed through the economical colloid lithography process, thus improving or maximizing the target productivity.

FIGS. 9 to 11 are cross-sectional views illustrating a method of manufacturing a target according to another embodiment of the inventive concept.

Referring to FIG. 9, the source layer 12 is formed on the substrate 10. The substrate 10 may include a bulk type silicon wafer or silicon bar. The source layer 12 may include a dielectric thin layer such as a silicon oxide layer or a silicon nitride layer. The source layer 12 may include a grid (not shown) formed by patterning the dielectric thin layer.

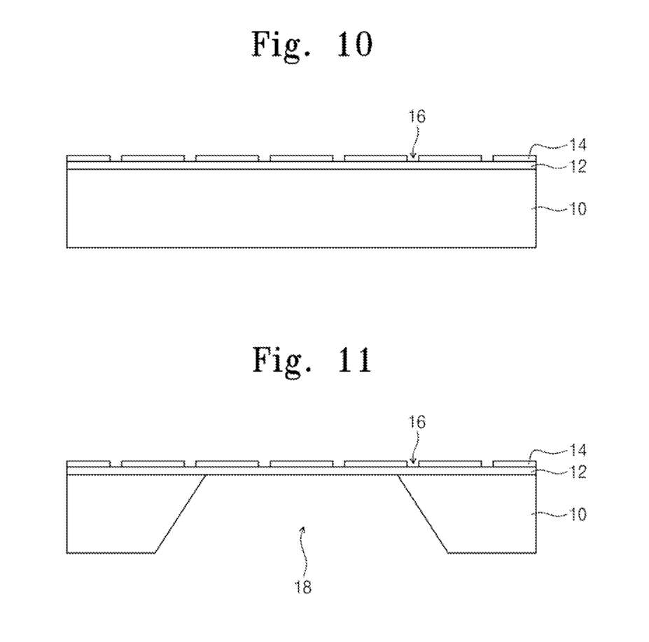

Referring to FIG. 10, the metal thin layer 14 including the nanoholes 16 partially exposing the source layer 12 is formed. The metal thin layer 14 may be formed through a sputtering process. The nanoholes 16 may be formed by patterning the metal thin layer 14 through an electron-beam lithography process or a photolithography process.

Referring to FIG. 11, a portion of the substrate 10 disposed under the metal thin layer 14 is removed to form the window 18 that partially exposes the source layer 12. The window 18 may be formed through an electron-beam lithography process, a photolithography process, or an etching process. The photolithography process forms a photoresist pattern (not shown), which selectively exposes the bottom surface of the substrate 10 to correspond to the window 18. The etching process selectively removes a portion the substrate 10 exposed through a photoresist pattern. The electron-beam lithography process selectively removes a portion of the substrate 10 remaining the bottom surface of the source layer 12 while the window 18 is formed.

Accordingly, the method according to the current embodiment can improve or maximize target productivity.

As described above, according to the embodiments of the inventive concept, a metal thin layer including nanoholes can emit charged particles of energy increased through plasmon resonance. The nanoholes may be formed through one of a colloid lithography process, a cleaning process, and an etching process, which are more economical than an electron-beam lithography process or a photolithography process.

According to the embodiments of the inventive concept, a method of manufacturing a target for generating charged particles, a target structure, and a treatment apparatus including the target structure can improve or maximize productivity.

The above-disclosed subject matter is to be considered illustrative, and not restrictive, and the appended claims are intended to cover all such modifications, enhancements, and other embodiments, which fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

What is claimed is:

1. A method of manufacturing a target of a treatment apparatus, comprising:
forming a source layer on a substrate;
forming balls on the source layer;
forming a metal thin layer on the source layer exposed between the balls; and
removing the balls to form holes in the metal thin layer so as to expose the source layer.

2. The method of claim 1, wherein the forming of the balls comprises a colloid lithograph process.

3. The method of claim 2, wherein the balls comprise at least one of a block copolymer and an amphiphilic surfactant.

4. The method of claim 3, wherein the balls are removed by a volatile solvent such as alcohol.

5. The method of claim 1, wherein the source layer comprises at least one of silicon, a dielectric and a carbon thin layer.

6. The method of claim 5, wherein the dielectric comprises a metal oxide layer, a silicon oxide layer, or a silicon nitride layer.

7. The method of claim 1, wherein the forming of the metal thin layer comprises a sputtering process or an electroplating process.

8. The method of claim 1, wherein the metal thin layer comprises at least one of gold, silver, and copper.

9. A target comprising:
a substrate;
a source layer disposed on the substrate; and
a metal thin layer comprising holes partially exposing the source layer.

10. The target of claim 9, wherein the metal thin layer comprises at least one of gold, silver, and copper.

11. The target of claim 9, wherein the holes of the metal thin layer have a size ranging from about 1 nm to about 1 μm.

12. The target of claim 9, wherein the substrate comprises a window that exposes the source layer or the metal thin layer to a lower side thereof.

13. The target of claim 12, wherein the window is provided in plurality to be arrayed in a matrix or in concentric circles within the substrate.

14. The target of claim 9, wherein the source layer comprises at least one of silicon, a dielectric, and a carbon thin layer.

15. The target of claim 14, wherein the dielectric comprises a metal oxide layer, a silicon oxide layer, or a silicon nitride layer.

16. The target of claim 14, wherein the carbon thin layer comprises at least one of graphene, graphite, fullerene, and a carbon nanotube.

17. A treatment apparatus comprising:
a light source providing a laser beam;
a guiding structure disposed at an incidence side of the laser beam provided by the light source; and
a target comprising a substrate fixed to the guiding structure, a source layer disposed on the substrate, and a metal thin layer comprising holes partially exposing the source layer,
wherein the metal thin layer accelerates, through surface plasmon resonance, charged particles generated in the source layer by the laser beam passing through the holes.

18. The treatment apparatus of claim 17, wherein the light source comprises a microwave laser.

19. The treatment apparatus of claim 17, wherein the holes of the metal thin layer have a size ranging from about 1 nm to about 1 μm.

20. The treatment apparatus of claim 17, wherein the metal thin layer comprises at least one of gold, silver, and copper.
